(19) United States
(12) Patent Application Publication
(10) Pub. No.: US 2002/0062501 A1
(43) Pub. Date: May 23, 2002

(54) Method of growth regulation treatment of turf grass and growth-regulated turf grass

(76) Inventor: Tokuzo Hirose, Ashiya-shi (JP)

Correspondence Address: George M. Kaplan, Esq., Dilworth & Barrese, LLP, 333 Earle Ovington Boulevard, Uniondale, NY, 11553, US

(21) Appl. No.: 09/734,861

(22) Filed: Dec. 12, 2000

Related U.S. Application Data
(63) Continuation-in-part of application No. 09/228,176, filed on Jan. 11, 1999, now abandoned.

(30) Foreign Application Priority Data
Jan. 13, 1997 (JP) P9-4091

(51) Int. Cl. A01H 5/00

(52) U.S. Cl. 800/294; 800/320; 800/290

(57) ABSTRACT

The object of the invention is to provide a treatment method of growth regulation for preventing wasteful growth of turf grass as well as growth-regulated turf grass. The invention relates to a treatment method for growth regulation of turf grass by introducing a rice-derived homeotic gene through a bacterium into a turf grass seed as well as to a growth-regulated turf grass produced by seeding and cultivating a turf grass seed obtained by this method.

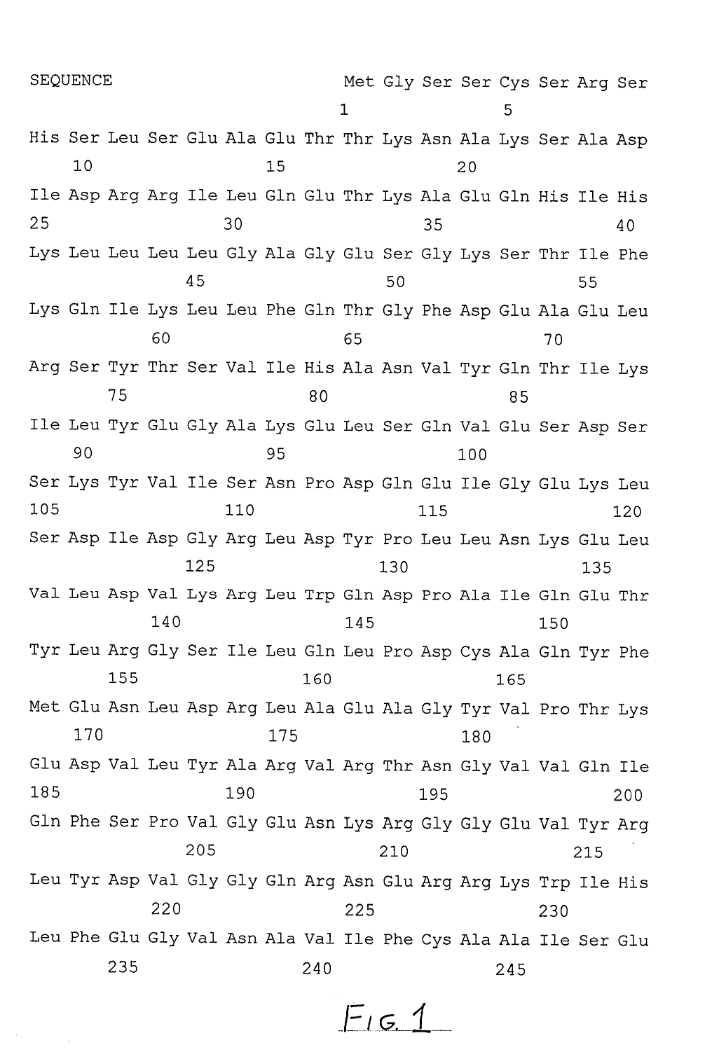

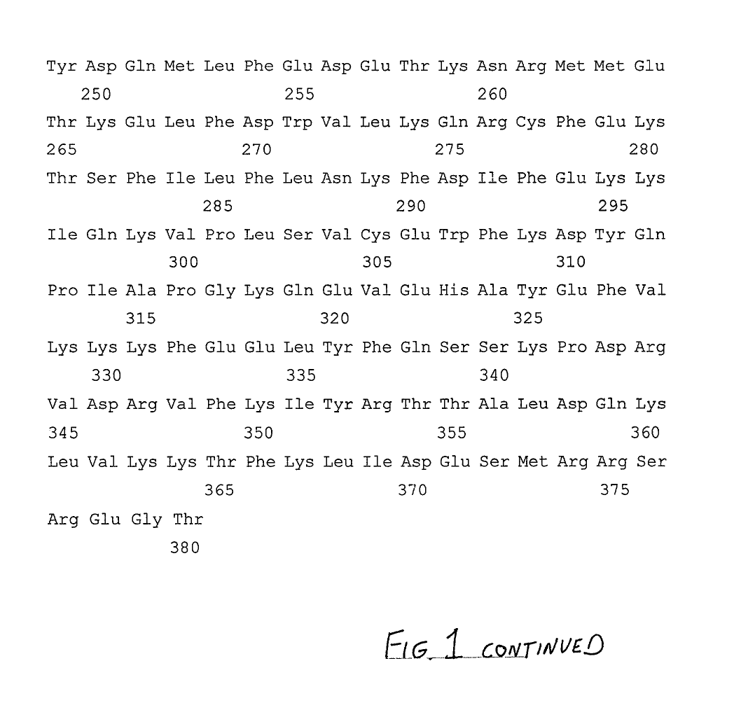

### BACKGROUND OF THE INVENTION

1. Field of the Invention

The present invention relates to a method of growth regulation treatment of turf grass and growth-regulated turf grass.

2. Description of the Related Art

Since many golf courses have been constructed since the golf boom at one time, golf courses recently have come to be surplus for a golf population. Accordingly, golf courses compete with one another in reducing play fees by reduction of expenditures.

In golf courses, much turf grass is used in the green, fairway etc. The turf grass will grow wastefully without trimming. Hence, the turf grass should always be trimmed. What is most troublesome in trimming of turf grass is to cut wastefully grown turf grass shortly to the same length. Some golf courses are sprayed with an agrochemical (dwarfing agent) to reduce the costs for turf grass cutting. However, in the case of spraying an agrochemical, agrochemical-containing water is released from the golf courses, which causes environmental pollution. Accordingly, the amount of an agrochemical sprayed has come to be limited.

### SUMMARY OF THE INVENTION

An object of the present invention is to provide a method of growth regulation treatment of turf grass for preventing wasteful growth of turf grass, and a growth-regulated turf grass.

The invention provides a method of growth regulation treatment of turf grass comprising introducing a homeotic gene through a bacterium into a turf grass seed.

Further, the invention provides a growth-regulated turf grass which is produced by sowing and cultivating a turf grass seed into which a homeotic gene is introduced through a bacterium.

According to the invention, a homeotic gene is introduced through a bacterium into a turf grass seed. The homeotic gene possesses a highly stored, characteristic sequence called “homeobox”, and it is considered to be involved in differentiation of tissues. A plant grows via repetition of successive cell division of its apical meristem, and particularly, growth of stems is promoted by the action of gibberellin, which is one of plant growth regulators. It is supposed that the homeotic gene in a plant expresses a DNA-binding gene regulatory protein to inhibit the action of gibberellin, thus inhibiting the growth of stems. The present inventors directed their attention to the fact that a turf grass is a grass of the Gramineae family, so they introduced a homeotic gene derived from a rice plant via a bacterium such as E. coli into a turf grass seed. The seed into which the homeotic gene has been introduced was sowed and the resulting turf grass was cultivated, and as a result, the turf grass was reduced in grass height to about ⅕ of usual turf grass. Accordingly, this growth-regulated turf grass can be used to reduce the frequency of mowing operation in golf courses to about ⅕, thereby reducing the costs for trimming of turf grass to about ⅕-⅓. Further, since the growth-regulated turf grass can be obtained without using any agrochemical such as a dwarfing agent, it does not adversely affect the environment.

In the invention it is preferable that the growth-regulated turf grass upwardly elongates in a nearly vertical direction up to a predetermined grass height.

According to the invention, the growth-regulated turf grasses having different grass heights from each other, obtained when the turf grass seeds having the homeotic gene introduced into them were sowed and cultivated, are divided into groups with grass heights in suitably predetermined ranges, and each group is subjected to pure cultivation whereby the grass height of the growth-regulated grass can previously be almost regulated. Accordingly, the growth-regulated turf grass having a grass height necessary depending on use can be selected and used, and the costs for trimming turf grass etc. can thereby be reduced. This technique can also apply similarly to the growth-regulated turf grasses obtained by introducing the homeotic gene into various phenotypes of turf grasses.

Further, in the invention it is preferable that the growth-regulated turf grass elongates upwardly in a nearly vertical direction up to a predetermined grass height and upon reaching the predetermined grass height, elongates downwardly so as to bend.

According to the invention, the growth-regulated turf grasses of which levels of regulation in growth were various were obtained at the stage when the turf grass seeds having the homeotic gene introduced into them were sowed and cultivated. Such individual differences of the growth-regulated turf grasses probably reflect differences in expression frequency and representation of a gene regulatory protein encoded by the introduced homeotic gene, or of differences in degree of inhibition of gibberellin. For example, some growth-regulated turf grasses grew upwardly in a nearly vertical direction up to a certain grass height. Some growth-regulated turf grasses grew upwardly in a neraly vertical direction, and upon reaching a certain grass height, they bent to grow downwardly. In these growth-regulated control turf grasses thus obtained, their grass heights were reduced in common as compared with usual turf grass. Accordingly, using such growth-regulated turf grasses subjected to pure cultivation in golf courses and the like to reduce the mowing operation thus reduces the costs for trimming of turf grass. Further, the growth-regulated turf grass does not have any adverse effect on the environment because it can be obtained without using any agrochemical such as a dwarfing agent.

In the invention it is preferable that the growth-regulated turf grass elongates in a nearly horizontal direction so as to reach a predetermined grass height.

Further, in the invention it is preferable that the growth-regulated turf grass elongates in a nearly vertical direction in the form of a curled coil up to a predetermined grass height.

According to the invention, the homeotic gene is introduced into various phenotypes of turf grasses to obtain the growth-regulated turf grasses of which levels of regulation in growth are various. For example, some growth-regulated turf grasses elongates in a nearly horizontal direction so as to reach a certain grass height. Some growth-regulated turf grasses grew in the form of curled coils up to a certain grass height. These growth-regulated turf grasses have their grass height reduced in common, as compared with usual turf grasses of the phenotypes. Accordingly, using such growth-regulated turf grasses which are subjected to pure cultivation, in golf courses and the like to reduce the mowing operation thus reduces the costs for trimming of turf grass. Further, the growth-regulated turf grass does not have any adverse effect on the environment because it can be obtained without using any agrochemical such as a dwarfing agent.

In the invention it is preferable that the growth-regulated turf grass has a lower rate of elongation in a nearly vertical direction than that of usual turf grass not subjected to the treatment method of the invention.

According to the invention, the growth-regulated turf grass obtained by introducing the homeotic gene into a seed had a reduced rate of elongation in the nearly vertical direction, as compared with usual turf grasses. This is probably because a gene regulatory protein encoded by the homeotic gene acts on the cell cycle of an apical meristem to reduce the rate of cell division. Accordingly, in the case where the growth-regulated turf grass is used as turf grass in golf courses etc., the frequency of operations such as a mowing operation etc. can be reduced owing to the reduced rate of elongation.

### DETAILED DESCRIPTION OF THE PREFERRED EMBODIMENTS

Hereinafter, one embodiment for carrying out the invention is described in detail.

The homeotic gene is cleaved with a restriction enzyme to give a linear DNA molecule, while a plasmid obtained from E. coli is cleaved with the same restriction enzyme to give a linear DNA molecule. Because of cleavage with the same restriction enzyme, the terminals of these DNA molecules have complementary base sequences to each other. A circular recombinant plasmid DNA into which the homeotic gene is inserted is formed by forming base pairs between the terminal of the homeotic gene and the terminal of the plasmid DNA and then covalently bonding the terminals by DNA ligase. E. coli was transfected with the circular recombinant plasmid DNA thus obtained and proliferated in a medium to give a large amount of E. coli carrying the homeotic gene.

The turf grass used was a Western turf grass. This is because a Japanese turf grass hardly fructifies and mainly grows via vegetative propagation. The E. coli was used to introduce the homeotic gene into a seed of the Western turf grass, and the seed was sowed to yield a growth-regulated turf grass. This growth-regulated turf grass was subjected to pure cultivation and its seed was sowed to produce a nearly 100% growth-regulated turf grass. Accordingly, the seed of the growth-regulated turf grass is regarded as being fixed.

While conventional Western turf grasses elongate wastefully to reach about 10 to 15 cm in 1 month in summer, the growth-regulated turf grass of one embodiment of the invention grows only about 3 to 5 cm in the same period. Accordingly, in the season (from April to November), conventional turf grasses were trimmed about 15 times in the green and about 10 times in fairways, but the growth-regulated turf grass of one embodiment of the invention can be used to reduce the frequency of trimming to about 4 times in the green and about 2 times in fairways, thus significantly reducing the costs for trimming. Further, the growth-regulated turf grass of the invention can be obtained merely by treating a seed, and E. coli has been sterilized, so that the turf grass does not have any adverse effect on the environment. Part of a highly elongate turf grass near the ground is hardly exposed to a wind and is often blighted by vermin and pests. The growth-regulated turf grass of the invention is free of such conditions, so the turf grass will not wither even if practically not cared for.

The levels of regulation in growth of the growth-regulated turf grasses produced by sowing turf grass seeds having the homeotic gene introduced into them were various. For example, some growth-regulated turf grasses elongated in a nearly vertical direction up to the certain grass height mentioned-above. Some growth-regulated turf grasses grew upwardly in a nearly vertical direction, and upon reaching a certain grass height, they bent to grow downwardly. Such individual differences of the growth-regulated turf grasses probably reflect differences in expression frequency and representation of a gene regulatory protein encoded by the introduced homeotic gene, or differences in degree of inhibition of gibberellin. lin.

When the homeotic gene was introduced by the treatment method of the invention into turf grasses of various phenotypes, growth-regulated turf grass of each phenotype having the grass height aforementioned was obtained. For example, some growth-regulated turf grasses elongated in a nearly horizontal direction so as to reach the grass height. Some growth-regulated turf grasses elongated in the form of curled coils so as to reach the grass height. By the technique of the invention, the growth-regulated turf grasses of various phenotypes can be obtained, and growth-regulated turf grasses of suitable phenotypes can be used depending on a wide variety of uses.

Further, the growth-regulated turf grasses produced by sowing the turf grass seeds having the homeotic gene introduced into them vary in their grass heights. These growth-regulated turf grasses with varying grass heights are selected for those with suitably determined lengths, for example, 0.5 cm, 1.0 cm, 1.5 cm and 2.0 cm, respectively, and the respective turf grasses are subjected to pure cultivation so that turf grasses having the previously set grass lengths of 0.5 cm, 1.0 cm, 1.5 cm and 2.0 cm, respectively, can be obtained. By producing the growth-regulated turf grasses which are regulated so as to elongate up to the predetermined grass height, the growth-regulated turf grass having a grass height necessary depending on use can be selected and used. In the case where the growth-regulated turf grass thus obtained is used as turf grass in e.g. golf courses, the costs for the mowing operation etc. can be reduced.

Further, the growth-regulated turf grass has a lower rate of elongation in the nearly vertical direction than that of usual turf grasses. This is probably because a gene regulatory protein encoded by the homeotic gene acts on the cell cycle of apical meristem to reduce the rate of cell division. Accordingly, in the case where the growth-regulated turf grass is used as turf grass in golf courses etc., the frequency of operations such as the mowing operation etc. can be reduced by virtue of the reduced rate of elongation.

The growth-regulated turf grass of the invention can be used not only in golf courses but also in parks, gardens, banks etc. so that the costs required for trimming of turf grass can be significantly reduced.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

What is claimed is:

1. A method of growth regulation treatment of turf grass comprising:
introducing a homeotic gene through a bacterium into a turf grass seed.

2. A growth-regulated turf grass which is produced by sowing and cultivating a turf grass seed into which a homeotic gene is introduced through a bacterium.

3. The growth-regulated turf grass of claim 2, the turf grass upwardly elongating in a nearly vertical direction up to a predetermined grass height.

4. The growth-regulated turf grass of claim 2, the growth-regulated turf grass elongating upwardly in a nearly vertical direction up to a predetermined grass height, the growth-regulated turf grass, upon reaching the predetermined grass height, elongating downwardly so as to bend.

5. The growth-regulated turf grass of claim 2, the growth-regulated turf grass elongating in a nearly horizontal direction so as to reach a predetermined grass height.

6. The growth-regulated turf grass of claim 2, the growth-regulated turf grass elongating in a nearly vertical direction in the form of a curled coil up to a predetermined grass height.

7. The growth-regulated turf grass of claim 2, the growth-regulated turf grass having a lower rate of elongation in a nearly vertical direction than that of usual turf grasses not subjected to the treatment method of claim 1.
